(19) United States
(12) Patent Application Publication
(10) Pub. No.: US 2015/0374600 A1
(43) Pub. Date: Dec. 31, 2015

(54) METHODS AND PREPARATIONS THAT TREAT HUMAN HAIR LOSS

(71) Applicants: Joseph F. Walsh, Sequim, WA (US); Sharon Kay Laska, Sequim, WA (US)

(72) Inventors: Joseph F. Walsh, Sequim, WA (US); Sharon Kay Laska, Sequim, WA (US)

(21) Appl. No.: 14/790,404

(22) Filed: Jul. 2, 2015

Related U.S. Application Data
(63) Continuation-in-part of application No. 12/703,932, filed on Feb. 11, 2010, now abandoned.
(60) Provisional application No. 61/207,420, filed on Feb. 11, 2009.

(51) Int. Cl. A61K 8/38

(52) U.S. Cl. CPC A61K 8/38; A61Q 7/00

(57) ABSTRACT

The current document is directed to methods that ameliorate and/or reverse human hair-loss. The methods render hair follicles accessible to treatment agents that remove blocking oil and cell remnants to allow additional treatment agents to enter the pilosebaceous units to kill or control microbes that disrupt hair growth by changing, inhibiting, or interrupting one or more biological functions of the pilosebaceous units.

### CROSS REFERENCE TO RELATED APPLICATIONS

This application is a continuation-in-part of U.S. application Ser. No. 12/703,932, filed Feb. 11, 2010, which claims benefit of U.S. Provisional Application No. 61/207,420, Feb. 11, 2009.

### TECHNICAL FIELD

The current document is directed to methods that treat hair loss.

### BACKGROUND

Although human hair seems most often viewed in terms of personal appearance and social status, human hair does provide many biological and physiological benefits, including protecting the skull and brain from injury, facilitating regulation of skull and brain temperature, and providing protection from UV radiation. Hair, stratum corneum, which is a layer of dead skin above living skin tissue of the scalp, and sebum, which refers to various scalp oils secreted by hair-follicle glands, are humans' first line of defense against UV radiation. UV radiation can damage genetic-information containing biopolymers, including deoxyribonucleic acid (“DNA”), within epidermal cells, potentially leading to melanoma and other cancers. Because hair and dead epidermal cells do not contain functional DNA, cannot undergo cell division, but absorb UV radiation, they provide good protection from the damaging effects of UV radiation.

The social aspects of hair often override the physiological benefits of hair for many people. People with thinning hair, or with significant hair loss referred to as “baldness,” particularly women, are often negatively regarded as less youthful and less attractive. Even mild psychological effects of baldness, such as diminished self-confidence, can have dramatic long term effects on a person's social, home and business lives.

Many treatments for human baldness have been sold over the years. These treatments were often sold with misleading or fraudulent claims. The situation became so serious that the Federal Drug Administration (“FDA”) intervened in the marketplace for hair-loss treatments, and now requires that a hair-loss-treatment product successfully undergo clinical trials and be approved by the FDA before it can be sold as a treatment or cure for baldness. To date, two products, minoxidil and finasteride, have been approved by the FDA for treatment of baldness. However, these products provide only a very modest, relatively short-term improvement for most users. Those who experience various degrees of hair loss, medical, pharmaceutical, and hair-products researchers and product developers, hair-care professionals, and many others continue to seek effective methods and preparations for treating hair loss.

### SUMMARY

The current document is directed to methods that ameliorate and/or reverse human hair-loss. The methods render hair follicles accessible to treatment agents that remove blocking oil and cell remnants to allow additional treatment agents to enter the pilosebaceous units to kill or control microbes that disrupt hair growth by changing, inhibiting, or interrupting one or more biological functions of the pilosebaceous units.

### BRIEF DESCRIPTION OF THE DRAWINGS

FIG. 1 illustrates the Norwood scale for assigning numerical values to represent the stage or degree of baldness in an individual.

FIG. 2 is a diagram of the pilosebaceous unit.

FIG. 3 provides an illustration of the hair-growth cycle.

FIG. 4A shows the chemical structure of benzoyl peroxide.

FIG. 4B shows the chemical structure for zinc pyrithione.

### DETAILED DESCRIPTION

Baldness is the lack of hair growth on the head, where hair once grew. Approximately 25% of men begin experiencing hair loss by age 30, increasing to about two-thirds of men who experience hair loss by age 60. Women experience increasing incidence of frontal hair loss with age. About 57% of women have frontal hair loss by age 80.

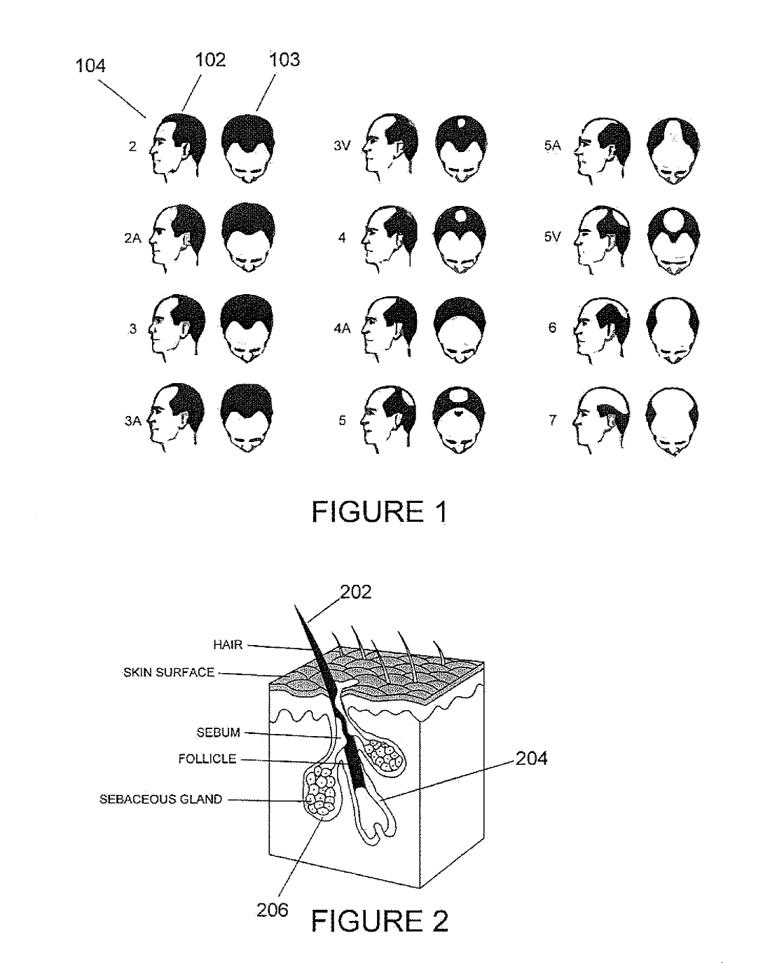

The most common form of baldness is a progressive thinning of the hair in men, called “male pattern baldness” (“MPB”). MPB starts as hair thinning on the lateral sides of the forehead and thinning on the top back of the head. The thinning areas expand over time and meet as one large bald area surrounded by a horse-shoe-shaped area of hair on the sides and back of head. For research purposes, stages of MPB are classified according to the Norwood Scale. FIG. 1 illustrates the Norwood scale for assigning numerical values to represent the stage or degree of baldness in an individual. In FIG. 1, the stages of baldness corresponding to a particular Norwood numerical value are illustrated by a pair of depictions of the extent of hair loss, such as the pair of depictions 102-103 associated with Norwood Scale value 2 (104 in FIG. 1). Female pattern baldness (“FPB”) is usually found in older women. However, FPB can also occur in teenage girls. Approximately 30% to 40% of women experience FPB in their lifetimes. FPB is associated with genetic factors similar to those associated with MPB, suggesting that it is a type of AGA. The general term for common baldness for both men and women is “androgenic alopecia” (“AGA”).

FIG. 2 is a diagram of the pilosebaceous unit. Each of the thousands of pilosebaceous units within a normal scalp produces a single hair. The pilosebaceous unit includes a hair shaft 202, a hair follicle 204, sebaceous glands which makes sebum 206, and an erector pili muscle which causes the hair to stand up when it contracts. The pilosebaceous unit also has tiny blood vessels that provide nutrients to the hair bulb. The sebaceous glands, located in pilosebaceous units, secrete sebum oil, which forms a protective layer of oil on the scalp. The sebum oil contains fatty acids with broad antimicrobial activity that can help protect the pilosebaceous from attack by microorganisms. Some microbials such as fungi and bacteria actually digest the antimicrobial sebum oils and use the sebum oil as a primary source of food. The oil digesting microbials and the antimicrobial sebum oils attain a delicate balance. This balance can be upset, resulting in plugged hair follicles, infection, and eventually hair-loss.

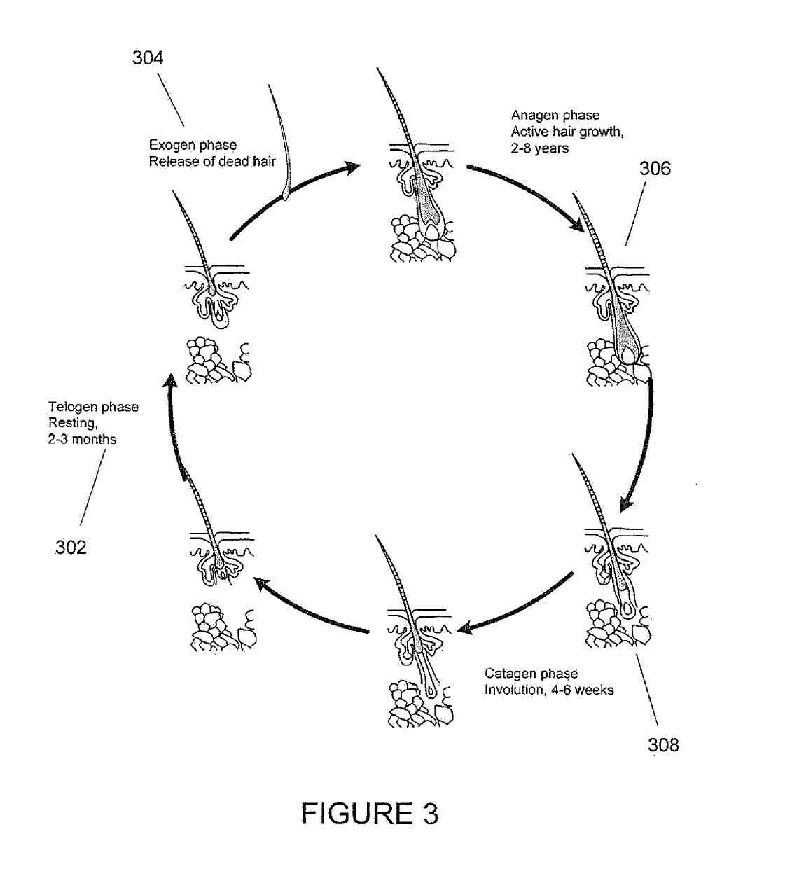

On average, about 100,000 hair follicles develop in the pre-natal scalp. Researchers currently believe that no hair follicles are created or lost after birth. Once developed, the follicles undergo a cycle of renewal in four phases. FIG. 3 provides an illustration of the hair-growth cycle. Phases of hair growth include: (1) a telogen phase 302, in which the hair follicle is resting, or dormant; (2) an exogen phase 304, in which a hair is released from the pilosebaceous unit as a new hair begins to grow within the hair follicle; (3) an anagen phase 306, characterized by active hair growth; and (4) a catagen phase 308, in which the hair follicle diminishes in size and regresses. When the number of hairs on an individual's scalp entering the catagen phase exceeds the number of hairs entering the anagen phase, the individual's hair is said to be thinning.

There are as many theories on the causes of baldness. It is generally agreed that baldness is governed by hereditary factors. However, there is much disagreement as to how those hereditary factors contribute to baldness. Testosterone and dehydroeplandrosterone (“DHEA”) are transformed into dihydrotestosterone (“DHT”) by 5-alpha reductase enzymes and hydroxysteroid-hydrogenase-isomerase enzymes in sebaceous glands of the pilosebaceous unit from which hair grows. DHT is a powerful sex hormone as well as a body and facial hair growth promoter, but can also have adverse affects on hair growth. One theory is that DHT may block hair receptors from receiving nutrients. Animal studies support the theory that testosterone and DHT are the main causes of AGA.

Certain studies have implicated yeast as a contributor or cause of baldness. One study concluded that Pityrosporum ovale (“Malassezia”), often present on the scalp of balding individuals, may contribute to baldness in both men and woman. The majority (89.92%) of balding subjects tested positive for Pityrosporum ovate scalp infection, while only 9.52% of non-balding subjects tested positive for Pityrosporum ovale scalp infection. Pityrosporum ovate digests sebum oil. Pityrosporum ovale creates an acidic environment favorable to other microorganism, such as propionibacterium acnes. Propionibacterium acnes are oxygen tolerant, anaerobic bacteria that prefer to grow in low oxygen environments deep inside hair follicles. Propionibacterium acnes use sebum (oil) as an energy source. In a plugged hair follicle, the low oxygen levels and accumulating sebum create an environment favorable for the growth of Propionibacterium acnes other microbials that contribute to hair-loss. Propionibacterium acnes and other hair-loss microbials also digest the oil in shampoos and conditioners.

The FDA has approved two preparations for treating baldness, minoxidil (“Rogain”) and finasteride (“Propecia. Minoxidil is a vasodilator, and is thought to stimulate hair growth by increasing blood flow and hence nutrients to hair roots. Finasteride is a 5-alpha reductase II inhibitor, and is thought to decrease conversion of dihydrotestosterone to DHA. Both provide modest, generally short-term increase in hair count within certain regions of the scalp, but neither comes close to reversing baldness, particularly the numerically larger Norwood-Scale stages of baldness. Researchers have tested the effectiveness of different combinations of minoxidil and pyrithione zinc, a fungicide, on hair density of men with MPB. A 6-month study of 200 patients tested various combinations of minoxidil and pyrithione zinc and found that hair counts were modestly and sustainably improved with daily use of a 1% pyrithione zinc shampoo. However, the benefit of 1% pyrithione-zinc shampoo, used alone, were not statistically significant.

Although the above-discussed research indicates that killing Pityrosporum ovate yeast on the surface of the scalp does not significantly reverse baldness, Pityrosporum ovate yeast, living within the pilosebaceous units inaccessible to topically applied fungicides, may nonetheless cause, contribute to, or facilitate AGA. The Pityrosporum ovate yeast may invade the pilosebaceous unit and disrupt the hair growth cycle. The Pityrosporum ovate yeast may also facilitate invasion of the pilosebaceous unit by one or more additional microorganisms, which in turn, disrupt the hair growth cycle in one or more ways, including: (1) secreting toxic wastes that damage hair; (2) disrupting DHT synthesis, binding of DHT to receptors, DHT catabolism, and other DHT-related processes; (3) disrupting hair follicles from receiving nutrients; (4) stimulating pilosebaceous units to produce hormones that signal their local and other, remote hair follicles to enter the resting cycle and/or fail to enter the anagen phase; (5) disrupting pilosebaceous-unit components of the immune system; (6) causing inflammation; and (7) digesting the oily, protective, sebum layer and leaving the pilosebaceous unit more exposed to microorganisms and other deleterious agents and conditions.

The current document is directed to methods that ameliorate and/or reverse human hair-loss as well as treatment solutions and preparations used in the methods. The methods render hair follicles accessible to treatment agents that remove blocking oil and cell remnants to allow additional treatment agents to enter the pilosebaceous units to kill or control microbes that disrupt hair growth by changing, inhibiting, or interrupting one or more biological functions of the pilosebaceous units. The hair-loss-amelioration and hair-loss-reversal methods disclosed in the current document use exfoliation agents, in come implementations combined with anti-inflammatory agents, to open the hair follicles and thereby render pilosebaceous units accessible to additional agents and substances, including anti-microbial agents, nutrients, and vasodilators.

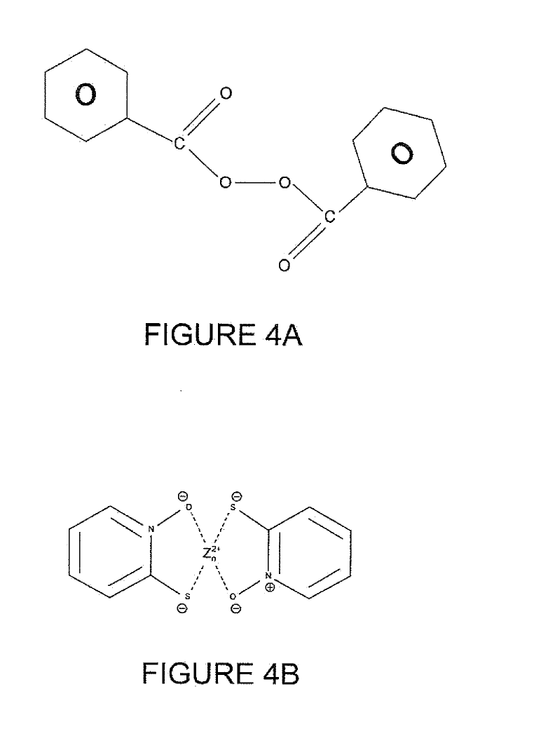

Benzoyl peroxide is an effective bactericide and exfoliation agent and that is used in acne treatments. FIG. 4A shows the chemical structure for benzoyl peroxide. The —O—O— bond is benzoyl peroxide is labile, and cleavage of this bond generates benzyol free radicals that damage bacterial biopolymers. FIG. 4 shows the structure of benzoyl peroxide. Benzoyl peroxide is unstable and has low solubility in water and only slight solubility in ethanol. Benzoyl peroxide is soluble in polyethylene glycol (“PEG”) and isopropyl myristate, but it is not stable in these pure solvents. The stability of benzoyl peroxide in PEG or isopropyl myristate is greatly increased upon the addition of water or ethanol.

Other exfoliation agents, including salicylic acid and D-limonene, are suitable agents for opening hair pores and rendering pilosebaceous units accessible to hair-loss-treatment agents. Salicylic acid and benzoyl peroxide work well together, allowing for a reduction in the concentration of benzoyl peroxide, which is a strong oxidizing agent. D-limonene acts as a solvent that opens pores by dissolving skin oils that bind dead skin cells together and plug the follicle opening as well as an anti-microbial agent, effective against bacteria, fungi, and other microbials

The antibiotics erythromycin and tetracycline are known to kill or control Propionibacterium acne and other bacteria. Additional bactericidal agents include: (1) various classes of antibiotic drugs, such as the beta-lactam antibiotics, penicillin and its derivatives, cephalosporins, monobactams, carbapenems, vanomycins, deptomycins, fluoroquinolones, metronidazoles, nitrofurantions, cotrimoxazoles, and aminoglycosidic antibiotics; small-molecule organic compounds, including ethanol, 1-propanol, 2-propanol, and other alcohols, chlorhexidine, brilliant green, chlorhexidine gluconate, phenol, and terpenes; (3) quaternary ammonium compounds, including benzalkonium chloride, cetyl trimethylammonium bromide, cetylpyridinium chloride, and benzethonium chloride; (4) inorganic compounds, including boric acid, hydrogen peroxide, iodine, sodium hypochlorite, and calcium hyopochlorite; and (5) heavy-metal compounds, such as mercurochrome.

Zinc pyrithione is an effective fungicide and antibacterial agent as well as an anti-inflammatory agent. FIG. 4B shows the chemical structure for zinc pyrithione. Zinc pyrthrione is sparingly soluble in water. It can be made soluble in water by adding an amine, such as ethanolamine, or by adding an alcohol, such as ethanol, in combination with a chelating agent, such as ethylenediaminetetraacetic acid (“EDTA”). Selenium sulfate is yet another suitable anti-microbial, anti-fungal and anti-bacteria agent. Other antifungal agents include: (1) polyene antifungals, including natamycin, filipin, nystatin, amphotericin B, and candicin; (2) imidazole and triazole antifungals, including miconazole, ketoconazole, clotrimazole, econazole, bifonazole, butoconazole, fenticonazole, isoconazole, oxiconazole, sertaconazole, sulconazole, tioconazole, fluconazole, itraconazole, isavuconazole, ravuconazole, posaconazole, voriconazole, and terconazole: (3) allylamines, including terbinafine, amorolfine, naftifine, and butenafine; (4) echinocandins, including anidulafungin, caspofungin, and micafungin; and (5) other antifungals, including benzoic acid, ciclopirox, tolnaftate, undecylenic acid, flucytosine, griseofulvin, haloprogin, allicin, tea tree oil, citronella oil, lemon grass, orange oil, palmarosa oil, patchouli, lemon myrtle, neem seed oil, coconut oil, zinc, selenium, and pentamidine dapsone atovaquone.

Thymol, also known as “2-isopropyl-5-methylphenol,” is a phenol with anti-microbial activity against bacteria, fungi, and other microbials. Thymol is also an anti-inflammatory agent, which facilitates opening of hair follicle, as well as. a penetrating agent, which facilitates enter of the thymol into hair follicles, where it kills bacteria and other microbials. Carvacrol, also known as “5-isopropyl-2-methylphenol,” is an phenol with anti-microbial properties, effective against both bacteria, fungi, and other microbials. Thymol and Carvacrol are compatible and work together synergistically. Thyme oil contains both Thymol and Carvacrol phenols.

Solutions that can be used in the currently disclosed methods include a bactericidal agent, combined with additional ingredients that can be topically applied to the scalp to introduce a sufficient quantity of the bactericidal agent into pilosebaceous units in order to kill or inhibit growth of microorganisms responsible for hair loss. Additionally, the solutions may include a bactericidal agent and an antifungal agent, combined with additional ingredients, that can be topically applied to the scalp to introduce a sufficient quantity of the bactericidal and antifungal agents to the scalp and sufficient quantity of the bactericidal agent into pilosebaceous units in order to kill or inhibit growth of microorganisms responsible for hair loss, and that additionally kill or inhibit yeast and other fungi that contribute to hair loss. Additionally, the solutions may include a bactericidal agent and an antifungal agent, combined with additional ingredients, that can be topically applied to the scalp to introduce a sufficient quantity of the bactericidal and antifungal agents into pilosebaceous units in order to kill or inhibit growth of microorganisms responsible for hair loss, and that additionally kill or inhibit yeast and other fungi that contribute to hair loss. Additionally, the solutions may include a broadband agent that is both a bactericidal and an antifungal agent, combined with additional ingredients, that can be topically applied to the scalp to introduce a sufficient quantity of this bactericidal and antifungal agent into pilosebaceous units in order to kill or inhibit growth of microorganisms responsible for hair loss. Additionally, the solutions may include a bactericidal agent, an antifungal agent, and a vasodilator combined with additional ingredients, that can be topically applied to the scalp to introduce a sufficient quantity of the bactericidal agent into pilosebaceous units in order to kill or inhibit growth of microorganisms responsible for hair loss, additionally kill or inhibit yeast and other fungi that contribute to hair loss, and promote increased delivery of nutrients to hair follicles.

Solutions that can be used in the currently disclosed methods additional include solutions that contain a bactericidal agent, bactericidal agent and antifungal agent, or bactericidal agent, antifungal agent, and vasodilator are combined with additional ingredients to create a shampoo that can be applied multiple times per day, on a daily basis, or at longer intervals, over the course of between several months and years, to kill or inhibit microorganisms within pilosebaceous units and restore hair growth to bald or thinning regions of a user's scalp. The additional ingredients may include water and other carriers and co-solvents, one or more surfactants, such as sodium lauryl sulfate, sodium laureth sulfate, and cocamidopropyl betaine, and other surfactants, inorganic salts, preservatives, fragrances, emulsifiers, such as polyethylene glycol, antioxidants, and other such ingredients.

Solutions that can be used in the currently disclosed methods include a bactericidal agent, bactericidal agent and antifungal agent, or bactericidal agent, antifungal agent, and vasodilator are combined with additional ingredients to create a leave-on solution or lotion that can be applied multiple times per day, on a daily basis, or at longer intervals, over the course of between several months and years, to kill or inhibit microorganisms within pilosebaceous units and restore hair growth to bald or thinning regions of a user's scalp. The additional ingredients may include water and other carriers and co-solvents, inorganic salts, preservatives, fragrances, emulsifiers, such as polyethylene glycol, antioxidants, and other such ingredients.

A shampoo is, in many ways, an ideal vehicle for treating hair loss. Shampoo is commonly and regularly used, for example. Treatments based on shampoos thus do not require a user to adapt to a new procedure and incur inconveniences and lifestyle changes. However, because a shampoo is in contact with the scalp for only a few minutes at a time, the shampoo needs to have a very efficient treatment-delivery system that carries the treatment directly into the pilosebaceous unit, where is can do the most good in the least period of time. A one-part hair-loss shampoo both treats hair-loss and conditions the hair. The hair-loss treatment has surfactant(s) for cleaning, agents to open hair pores, and hair-loss treatment to be delivered to the pilosebaceous unit. Silicon-based conditioning agents are used, such as dimethicone suspended in glycol disterate, because silicon based conditioners do not provide nutrients to microbials that are major contributors to hair-loss in humans. A two-part hair-loss shampoo includes: (1) a first-step solution that opens the pilosebaceous unit and cleans or flushes out micro-organisms, such as bacteria and fungi, to allow hair-loss treatments to enter; and (2) a second-step solution that treats hair-loss and conditions hair, including low concentrations (<2%) of thymol and carvacrol phenols or other anti-microbial agents such as pyrithione zinc and selenium sulfate.

The current document is directed to a variety of different methods that incorporate the regular use of low concentrations of benzoyl peroxide and other combinations of ingredients, discussed above. The methods may introduce benzoyl peroxide in shampoos, conditioners, and/or leave-on solutions, and include the use of one-part and two-part shampoos and treatments. The benzoyl-peroxide concentration in applied solutions may be as high as 3%, for treatment solutions used relatively infrequently, such as one or two times a month, but is generally 1% or less in solutions applied more frequently to the scalp. The benzoyl peroxide provides effective exfoliation and anti-bacterial and anti-fungal activities, and effectively renders pilosebaceous units accessible both to benzoyl peroxide, itself, as well as to minoxidal and additional agents and substances. As discussed above, various combinations of one or more of salicylic acid, D-limonene, thymol and carvacrol phenols, pyrithione zinc, and selenium sulfate are also employed in treatment solutions to facilitate opening and cleaning of hair follicles and killing or control of bacteria and fungi in the pilosebaceous units. Although the present invention has been described in terms of a particular embodiment, it is not intended that the invention be limited to this embodiment. Modifications within the spirit of the invention will be apparent to those skilled in the art. For example, additional implementations use shampoos and leave-on solutions in which a different antibacterial agent is substituted for, or added in combination with, benzoyl peroxide, a different antifungal agent is substituted for, or added in combination with, zinc pyrithione, and a different vasodilator is substituted for, or added in combination with, minoxidil. These different antibacterial agents, antifungal agents, and vasodilators may be selected from among the above-mentioned compounds and substances or may be selected from additional antibacterial agents, antifungal agents, and vasodilators such that, when combined with a solvent and other ingredients of a hair-loss-treatment shampoo or leave-on solution or lotion, an effective concentration of the antibacterial agents, antifungal agents, and/or vasodilators is produced in the pilosebaceous units of the scalp to which the solutions are applied.

The foregoing description, for purposes of explanation, used specific nomenclature to provide a thorough understanding of the invention. However, it will be apparent to one skilled in the art that the specific details are not required in order to practice the invention. The foregoing descriptions of specific embodiments of the present invention are presented for purposes of illustration and description. They are not intended to be exhaustive of or to limit the invention to the precise forms disclosed. Many modifications and variations are possible in view of the above teachings. The embodiments are shown and described in order to best explain the principles of the invention and its practical applications, to thereby enable others skilled in the art to best utilize the invention and various embodiments with various modifications as are suited to the particular use contemplated. It is intended that the scope of the invention be defined by the following claims and their equivalents:

1. A method that ameliorates and/or reverses human baldness, the method comprising:
providing a solution that includes benzoyl peroxide at a concentration of 3% or less; and
applying the solution to a patient's scalp with a frequency of between twice a day and once a month.

2. A preparation that ameliorates and/or reverses human baldness, the preparation comprising:
water;
3% or less of benzoyl peroxide;
salicylic acid; and
one or more additional substances selected from among
D-limonene,
minoxidil,
thymol,
carvacrol,
pyrithione zinc, and
selenium sulfate.
